(19) United States
(12) Patent Application Publication
(10) Pub. No.: US 2023/0000390 A1
(43) Pub. Date: Jan. 5, 2023

(54) LOAD MEASUREMENT SYSTEM, WALKING TRAINING SYSTEM, LOAD MEASUREMENT METHOD, AND PROGRAM

(71) Applicant: TOYOTA JIDOSHA KABUSHIKI KAISHA, Toyota-shi (JP)

(72) Inventor: Taiga Matsumoto, Nagoya-shi (JP)

(73) Assignee: TOYOTA JIDOSHA KABUSHIKI KAISHA, Toyota-shi (JP)

(21) Appl. No.: 17/713,561

(22) Filed: Apr. 5, 2022

(30) Foreign Application Priority Data
Jun. 30, 2021 (JP) 2021-108412

(51) Int. Cl. A61B 5/103; A61H 1/02

(52) U.S. Cl. CPC A61B 5/1038; A61H 1/0262; A61B 2505/09; A61B 2562/0252

(57) ABSTRACT

A load measurement device includes an acquisition unit, a load calculation unit, and an output unit. The acquisition unit acquires sensor output information of a load distribution sensor that detects a load distribution received from a sole of a subject. The load calculation unit calculates a total load value, based on the sensor output information and geometric information of a load region specified based on the sensor output information, the geometric information including at least a perimeter of the load region in a horizontal direction. The output unit outputs the total load value.

### CROSS-REFERENCE TO RELATED APPLICATION

This application claims priority to Japanese Patent Application No. 2021-108412 filed on Jun. 30, 2021, incorporated herein by reference in its entirety.

### BACKGROUND

### 1. Technical Field

The present disclosure relates to a load measurement system, a walking training system, a load measurement method, and a program.

### 2. Description of Related Art

A rehabilitation (rehab) training system has been developed for rehab patients to train their walking motion. For example, Japanese Unexamined Patent Application Publication No. 2017-035220 (JP 2017-035220 A) discloses a walking training device that assists the motion of joints of a patient via a walking assist device in accordance with the walking state of a user who is wearing the walking assist device on his/her leg. Such a rehabilitation training system estimates the walking state of the patient based on the load distribution acquired from the load distribution sensor sheet arranged under the belt of the treadmill, and assists the movement of the patient's joints.

### SUMMARY

Here, the load distribution sensor sheet includes a viscoelastic sheet. In the load distribution sensor including the viscoelastic body sheet, there is a problem that a part of the input load is converted into stress in the sliding direction (horizontal direction), and the measurement accuracy of the load value in the vertical direction is decreased. Especially when applied to a rehabilitation training system, since a low-friction sheet and a belt are stacked on the load distribution sensor sheet, the input load further flows in the horizontal direction due to the influence of the belt and the low-friction sheet, and there is a pronounced decrease in the measurement accuracy.

The present disclosure has been made to solve such problems, and provides a load measurement system, a walking training system, a load measurement method, and a program that improve the measurement accuracy of a load value.

A load measurement system according to one aspect of the present disclosure includes an acquisition unit, a load calculation unit, and an output unit. The acquisition unit acquires sensor output information of a load distribution sensor that detects a load distribution received from a sole of a subject. The load calculation unit calculates a total load value, based on the sensor output information and geometric information of a load region that is specified based on the sensor output information, the geometric information including at least a perimeter of the load region in a horizontal direction. The output unit outputs the total load value.

As a result, it is possible to acquire a load value in the vertical direction in consideration of the load amount that has flowed in the horizontal direction. Therefore, the measurement accuracy of the load value is improved.

Here, the load measurement system may further include a geometric calculation unit that specifies a region in which an output value of a cell included in the load distribution sensor is equal to or higher than a predetermined value, and that calculates the geometric information of the load region.

This makes it possible to easily calculate the geometric information by using the geometric features of the cell.

The load calculation unit may calculate the total load value by using a map that associates the geometric information with a total of an output value of a cell included in the load region and the total load value.

Thus, the load measurement system can easily convert the sensor output information into the total load value by using the geometric information.

Further, the load calculation unit may use a map that associates the geometric information with output values of cells included in the load region and pressure values to calculate a pressure value for each of the cells included in the load distribution sensor, and the load distribution sensor may calculate the total load value based on the pressure value of each of the cells.

Thus, the load measurement system can easily convert the sensor output information into the total load value by using the geometric information. Further, since the load measurement system converts the output value into the pressure value one cell at a time, the load measurement accuracy is further improved.

Further, the load calculation unit may calculate the total load value by using a trained load estimation model in which an input is an output value of a cell included in the load region, or a total of the output value of the cell, and in which an output is the total load value.

As a result, the load measurement system can easily estimate the total load value even for an unknown combination of the geometric information and the sensor output information.

A walking training system according to one aspect of the present disclosure includes the load measurement system, the load distribution sensor, a moving body, and a control device. The control device controls extension of a leg robot attached to at least one leg of the subject, the subject walking on a walking surface provided on the moving body, based on the total load value output by the load measurement system. As a result, it is possible to acquire a load value in the vertical direction in consideration of the load amount that has flowed in the horizontal direction. Thus, the measurement accuracy of the load value is improved, and the control device can appropriately control the extension of the leg robot.

A load measurement method according to one aspect of the present disclosure includes: acquiring sensor output information of a load distribution sensor that detects a load distribution received from a sole of a subject; calculating a total load value, based on the sensor output information and geometric information of a load region specified based on the sensor output information, the geometric information including at least a perimeter of the load region in a horizontal direction; and outputting the total load value.

As a result, it is possible to acquire a load value in the vertical direction in consideration of the load amount that has flowed in the horizontal direction. Therefore, the measurement accuracy of the load value is improved.

A program according to one aspect of the present disclosure causes a computer to execute: an acquisition process of acquiring sensor output information of a load distribution sensor that detects a load distribution received from a sole of a subject; a load calculation process of calculating a total load value, based on the sensor output information and geometric information of a load region specified based on the sensor output information the geometric information including at least a perimeter of the load region in a horizontal direction; and an output process of outputting the total load value. As a result, it is possible to acquire a load value in the vertical direction in consideration of the load amount that has flowed in the horizontal direction. Therefore, the measurement accuracy of the load value is improved.

The present disclosure can provide a load measurement system, a walking training system, a load measurement method, and a program with which the measurement accuracy of a load value is improved.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic perspective view of a walking training system according to a first embodiment;

FIG. 2 is a schematic perspective view showing a configuration example of a walking assist device;

FIG. 3 shows a side view and a top view of a treadmill according to the first embodiment;

FIG. 4 is a front view of a region A according to the first embodiment;

FIG. 5 is a front view of the region A for explaining a problem of the embodiment;

FIG. 6 is a diagram showing an example of sensor output information;

FIG. 7 is a block diagram showing a schematic configuration of a load measurement device according to the first embodiment;

FIG. 8 is a diagram for explaining a process of calculating geometric information according to the first embodiment;

FIG. 9 is a diagram showing an example of a conversion map according to the first embodiment;

FIG. 10 is a flowchart showing a procedure of a load measurement method according to the first embodiment;

FIG. 11 is a diagram showing an example of a conversion map according to a second embodiment;

FIG. 12 is a flowchart showing a procedure of a total load value calculation process in a load measurement method according to the second embodiment;

FIG. 13 is a block diagram showing a schematic configuration of a load measurement device according to a third embodiment; and

FIG. 14 is a schematic configuration diagram of a computer that is used as the load measurement device and a system control unit according to the first to third embodiments.

### DETAILED DESCRIPTION OF EMBODIMENTS

Hereinafter, the present disclosure will be described through embodiments, but the disclosure according to the claims is not limited to the following embodiments. Moreover, not all of the configurations described in the embodiments are indispensable as means for solving the problem. For the sake of clarity, omission and simplification are made in the following description and drawings as appropriate.

### First Embodiment

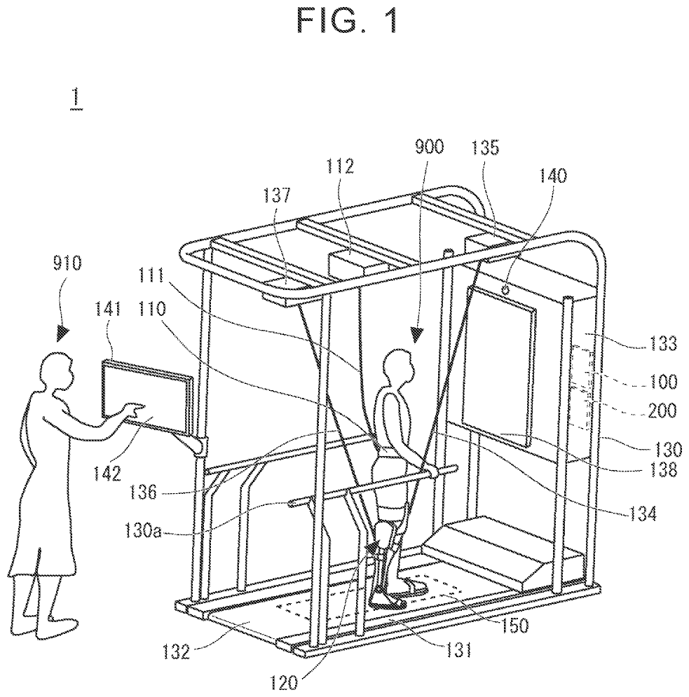

Hereinafter, a first embodiment of the present disclosure will be described. FIG. 1 is a schematic perspective view of a walking training system 1 according to the first embodiment. The walking training system 1 is an example of a system to which a load measurement device (also referred to as a load measurement system) according to the first embodiment can be applied. The walking training system 1 is a system for a trainee 900 who is a hemiplegic patient suffering from paralysis in one leg to perform walking training. The trainee 900 is also referred to as a subject. The up-down direction, the right-left direction, and the front-rear direction in the following description represent directions with the direction of the trainee 900 as a reference.

The walking training system 1 mainly includes a control panel 133 attached to a frame 130 constituting the entire skeleton, a treadmill 131 on which the trainee 900 walks, and a walking assist device 120 attached to at least one leg of the trainee 900. In the first embodiment, at least one leg is the affected leg that is the leg of the trainee 900 on the paralyzed side.

The frame 130 is provided to stand on the treadmill 131 installed on the floor. The treadmill 131 is a moving body that rotates a ring-shaped belt 132 with a motor (not shown). Thus, the belt 132 travels along the orbit. The treadmill 131 is a device that prompts the trainee 900 to walk. The trainee 900 who performs walking training rides on the belt 132 and attempts a walking motion with respect to the walking surface provided on the belt 132.

The frame 130 supports the control panel 133 and a training monitor 138. The control panel 133 accommodates a load measurement device 100 and a system control unit 200. The load measurement device 100 is a computer device that measures the total load value of the load region received by the sole of the trainee 900 based on the measurement result of the sensor. The system control unit 200 is also referred to as a control device, and is a computer device that controls sensors and motors. For example, the system control unit 200 controls the extension of the walking assist device 120 based on the total load value measured by the load measurement device 100.

The training monitor 138 is a display device that presents information on training and measurement to the trainee 900. The training monitor 138 is, for example, a liquid crystal panel. The training monitor 138 is installed such that the trainee 900 can visually recognize the training monitor 138 while walking on the belt 132 of the treadmill 131.

Further, the frame 130 supports a front tension unit 135 at the front of the overhead portion of the trainee 900, a harness tension unit 112 at the overhead portion, and a rear tension unit 137 at the rear of the overhead portion. The frame 130 may include handrails 130a for the trainee 900 to grab.

The camera 140 is a front camera unit that takes an image of the trainee 900 at such an angle of view that the gait of the trainee 900 can be recognized from the front. The camera 140 may include a side camera unit that takes an image of the trainee 900 at such an angle of view that the gait of the trainee 900 can be recognized from the side. The camera 140 in the present embodiment includes a set of a lens and an imaging element that provides such an angle of view that the whole body including the head of the trainee 900 standing on the belt 132 can be captured. The imaging element is, for example, a complementary metal oxide semiconductor (CMOS) image sensor, and converts an optical image on an image plane into an image signal. The camera 140 is installed near the training monitor 138 so as to face the trainee 900. When the camera 140 includes the side camera unit, the side camera unit may be installed on the handrail 130a so as to capture the trainee 900 from the side.

One end of a front wire 134 is connected to a winding mechanism of the front tension unit 135, and the other end is connected to the walking assist device 120. The winding mechanism of the front tension unit 135 winds and unwinds the front wire 134 in accordance with the movement of the affected leg, by turning on and off a motor (not shown) following the instruction of the system control unit 200. Similarly, one end of a rear wire 136 is connected to a winding mechanism of the rear tension unit 137, and the other end is connected to the walking assist device 120. The winding mechanism of the rear tension unit 137 winds and unwinds the rear wire 136 in accordance with the movement of the affected leg, by turning on and off a motor (not shown) following the instruction of the system control unit 200. With such a coordinated operation of the front tension unit 135 and the rear tension unit 137, the load of the walking assist device 120 is offset so as not to be a burden on the affected leg, and further, the forward swing motion of the affected leg is assisted in accordance with the degree of the setting.

An operator 910 who is a training assistant sets the assist level high, for a trainee who has severe paralysis. The operator 910 is a physiotherapist or a doctor who has the authority to select, correct, and add the setting items of the walking training system 1. When the assist level is set high, the front tension unit 135 winds up the front wire 134 with a relatively large force in accordance with the forward swing timing of the affected leg. As the training progresses and assistance becomes no longer needed, the operator sets the assist level to the minimum. When the assist level is set to the minimum, the front tension unit 135 winds up the front wire 134 with a force to cancel the weight of the walking assist device 120 in accordance with the forward swing timing of the affected leg.

The walking training system 1 includes a safety device including, as main components, a safety brace 110, a harness wire 111, and the harness tension unit 112. The safety brace 110 is a belt wrapped around the abdomen of the trainee 900 and is fixed to the waist by, for example, a hook-and-loop fastener. One end of the harness wire 111 is connected to the safety brace 110, and the other end is connected to the winding mechanism of the harness tension unit 112. The winding mechanism of the harness tension unit 112 winds and unwinds the harness wire 111 by turning on and off a motor (not shown). With such a configuration, when the trainee 900 significantly loses his/her posture, the safety device winds up the harness wire 111 following the instruction of the system control unit 200 that has detected the movement, and supports the upper body of the trainee 900 with the safety brace 110.

A management monitor 141 is attached to the frame 130 and is a display device for the operator 910 to monitor and operate. The management monitor 141 is, for example, a liquid crystal panel, and a touch panel is superimposed on the surface of the management monitor 141 as an example of an input portion 142. The management monitor 141 presents various menu items related to settings of training and measurement, various parameter values at the time of training and measurement, measurement results during training, and the like. The operator 910 selects, corrects, or adds a setting item via the input portion 142 such as a touch panel or a keyboard (not shown). The management monitor 141 is installed at a position where the trainee 900 cannot visually recognize the display from the training trial position on the treadmill 131. A support portion that supports the management monitor 141 may have a rotation mechanism that inverts the display surface in order to cope with the case where the operator 910 intentionally shows the display screen to the trainee 900.

The walking assist device 120 is attached to the affected leg of the trainee 900 and assists the trainee 900 in walking by reducing the load of extension and bending on the knee joint of the affected leg. The walking assist device 120 transmits data on the leg movement acquired through the walking training to the system control unit 200, and drives the joint portion following the instruction from the system control unit 200. The walking assist device 120 can also be connected, via a wire or the like, to a hip joint (a connecting member including a rotating portion) attached to the safety brace 110 that is a part of the fall prevention harness device.

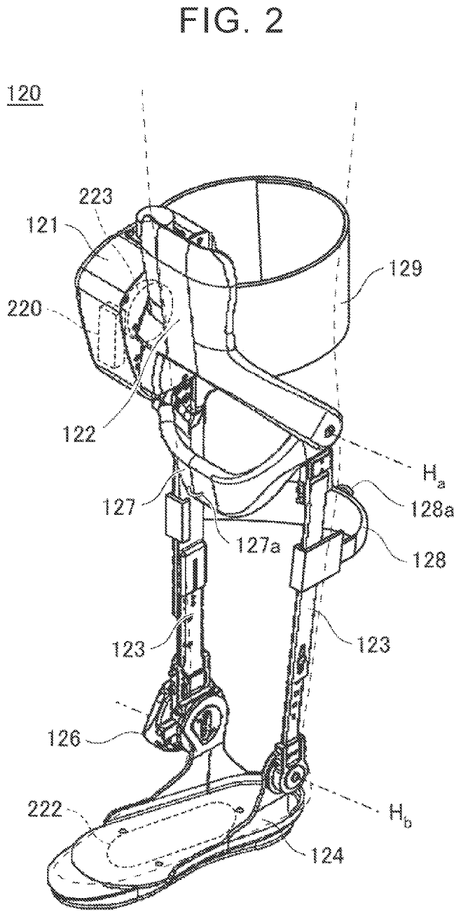

FIG. 2 is a schematic perspective view showing a configuration example of the walking assist device 120. The walking assist device 120 mainly includes a control unit 121 and a plurality of frames that supports various parts of the affected leg. The walking assist device 120 is also referred to as a leg robot.

The control unit 121 includes an auxiliary control unit 220 that controls the walking assist device 120, and also includes a motor (not shown) that generates a driving force for assisting the extension motion and the bending motion of the knee joint. The frames that support various parts of the affected leg include an upper leg frame 122 and lower leg frames 123 that are pivotably connected to the upper leg frame 122. The frames further include a foot flat frame 124 pivotably connected to the lower leg frames 123, a front connecting frame 127 for connecting the front wire 134, and a rear connecting frame 128 for connecting the rear wire 136.

The upper leg frame 122 and the lower leg frames 123 pivot relative to each other around a hinge axis Hₐ shown in the figure. The motor of the control unit 121 rotates following the instruction of the auxiliary control unit 220 to force the upper leg frame 122 and the lower leg frames 123 to relatively open and close around the hinge axis Hₐ. An angle sensor 223 accommodated in the control unit 121 is, for example, a rotary encoder, and detects the angle between the upper leg frame 122 and the lower leg frames 123 around the hinge axis Hₐ. The lower leg frames 123 and the foot flat frame 124 pivot relative to each other around a hinge axis H_{b} shown in the figure. The relative pivot angle range is adjusted in advance by an adjusting mechanism 126.

The front connecting frame 127 is provided so as to extend in the right-left direction on the front side of the upper leg and connect to the upper leg frame 122 at both ends. The front connecting frame 127 is further provided with a connecting hook 127a for connecting the front wire 134, around the center in the right-left direction. The rear connecting frame 128 is provided so as to extend in the right-left direction on the rear side of the lower leg and connect to the lower leg frames 123 at both ends. Further, the rear connecting frame 128 is provided with a connecting hook 128a for connecting the rear wire 136, around the center in the right-left direction.

The upper leg frame 122 is provided with an upper leg belt 129. The upper leg belt 129 is a belt integrally provided on the upper leg frame, and is wrapped around the upper leg portion of the affected leg to fix the upper leg frame 122 to the upper leg portion. This suppresses the entire walking assist device 120 from shifting with respect to the legs of the trainee 900.

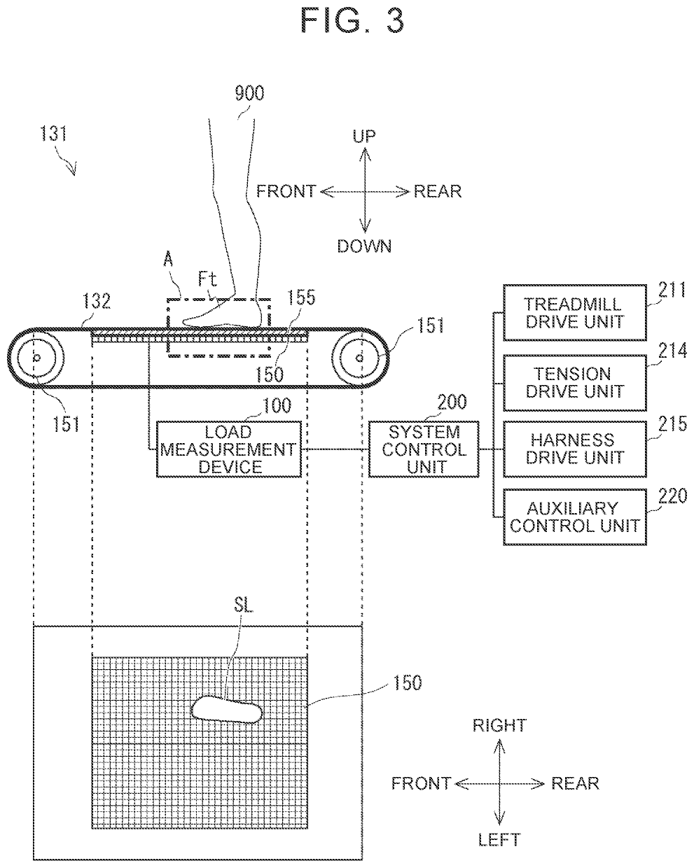

FIG. 3 shows a side view and a top view of the treadmill according to the first embodiment. The treadmill 131 includes at least a ring-shaped belt 132, a pulley 151, and a motor (not shown).

Further, via a low friction sheet 155, a load distribution sensor 150 is arranged on the lower side of the belt 132 of the treadmill 131, that is, on an opposite side of the belt 132 from the surface on which the trainee 900 is boarded. The load distribution sensor 150 is fixed to the body of the treadmill 131 so as not to move together with the belt 132.

The load distribution sensor 150 is a load distribution sensor sheet including a plurality of cells in which the cells are pressure detection points. The cells are arranged in a matrix so as to be parallel with a walking surface W (mounting surface) that supports a sole Ft of the trainee 900 in the standing state. Further, the load distribution sensor 150 is disposed toward the center of the walking surface Win the right-left direction that is orthogonal to the walking front-rear direction. The walking front-rear direction is a direction parallel to the traveling direction of the belt 132. By using output values of the cells, the load distribution sensor 150 can detect the magnitude and the distribution of the vertical load received from the sole Ft of the trainee 900. Thereby, the load distribution sensor 150 detects the position of the sole Ft of the trainee 900 in the standing state and the distribution of the load received from the sole Ft of the trainee 900, via the belt 132. Here, the region where the load is detected when the sole Ft is in contact with the ground is called a load region SL.

The load distribution sensor 150 is connected to the load measurement device 100. The load measurement device 100 acquires the sensor output information output from the load distribution sensor 150, and calculates the total load value of the load region based on the sensor output information. The load measurement device 100 supplies the information of the measured total load value to the system control unit 200.

The system control unit 200 controls various drive units based on the total load value. For example, the system control unit 200 is connected to a treadmill drive unit 211, a tension drive unit 214, a harness drive unit 215, and the auxiliary control unit 220 of the walking assist device 120 by wire or wirelessly. The system control unit 200 transmits drive signals to the treadmill drive unit 211, the tension drive unit 214, and the harness drive unit 215, and transmits a control signal to the auxiliary control unit 220.

The treadmill drive unit 211 includes the above motor for rotating the belt 132 of the treadmill 131 and a drive circuit thereof. The system control unit 200 performs rotation control of the belt 132 by transmitting a drive signal to the treadmill drive unit 211.

The system control unit 200 adjusts the rotation speed of the belt 132 in accordance with, for example, the walking speed set by the operator 910. Alternatively, the system control unit 200 adjusts the rotation speed of the belt 132 in accordance with the information of the total load value output from the load measurement device 100.

The tension drive unit 214 includes a motor for tensioning the front wire 134 and a drive circuit thereof that are provided in the front tension unit 135, and a motor for tensioning the rear wire 136 and a drive circuit thereof that are provided in the rear tension unit 137. The system control unit 200 controls the winding of the front wire 134 and the winding of the rear wire 136 by transmitting a drive signal to the tension drive unit 214. Further, the system control unit 200 controls the tensile force of each wire by controlling the driving torque of the motor as well as the winding operation. Further, the system control unit 200 identifies the timing at which the affected leg switches from the standing state to the swinging state based on the total load value output from the load measurement device 100, and increases or decreases the tensile force of each wire in synchronization with that timing, thereby assisting the motion of the affected leg.

The harness drive unit 215 includes a motor for tensioning the harness wire 111 and a drive circuit thereof that are provided in the harness tension unit 112. The system control unit 200 controls the winding of the harness wire 111 and the tensile force of the harness wire 111 by transmitting a drive signal to the harness drive unit 215. For example, when the trainee 900 is predicted to fall, the system control unit 200 winds up the harness wire 111 by a certain amount to suppress the trainee from falling.

The auxiliary control unit 220 is, for example, a microprocessor unit (MPU), and performs control of the walking assist device 120 by executing a control program provided from the system control unit 200. Moreover, the auxiliary control unit 220 notifies the system control unit 200 of the state of the walking assist device 120. Further, the auxiliary control unit 220 receives a command from the system control unit 200 based on the total load value and performs control of starting, stopping, and the like of the walking assist device 120.

The auxiliary control unit 220 transmits a drive signal to the joint drive unit including the motor of the control unit 121 and the drive circuit thereof, to force the upper leg frame 122 and the lower leg frames 123 to relatively open and close around the hinge axis Hₐ. Such motions assist the extension motion and the bending motion of the knee and suppress knee collapse. The auxiliary control unit 220 receives a detection signal from an angle sensor (not shown) that detects the angle between the upper leg frame 122 and the lower leg frames 123 around the hinge axis Hₐ, and calculates the opening angle of the knee joint.

Note that FIG. 3 shows the sole Ft of the trainee 900 and the region A around the load distribution sensor 150.

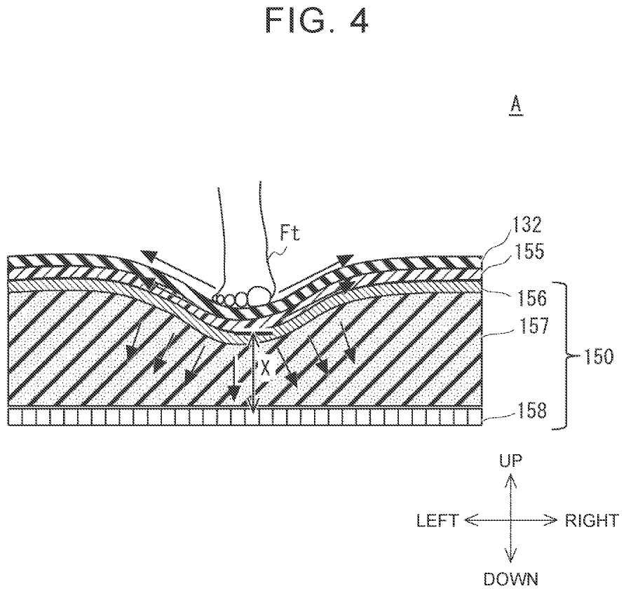

Here, FIG. 4 is a front view of the region A according to the first embodiment. The load distribution sensor 150 has a structure in which a coil portion 158, a cushion 157, and a metal sheet 156 are laminated in this order from the bottom. The coil portion 158 includes cells in a matrix shape, each of which functions as a pressure detection point. When a load is received from the sole Ft, a distance X between the metal sheet 156 and the coil portion 158 becomes shorter and thus, electromagnetic coupling occurs. The load distribution sensor 150 detects the coupling coefficient of each cell corresponding to the displacement of the distance X, and outputs the sensor output information including the pressure corresponding to the coupling coefficient of each cell as an output value (for example, a voltage value).

Here, the low friction sheet 155 and the belt 132 are laminated on the load distribution sensor 150. Due to such a multi-layer structure, in the region A, when the sole Ft is in contact with the ground, the seat is dragged, the upper and lower layers are strained, and the cushion 157 is strained. As a result, a part of the load input in the vertical direction is converted into stress due to the deformation in the horizontal direction.

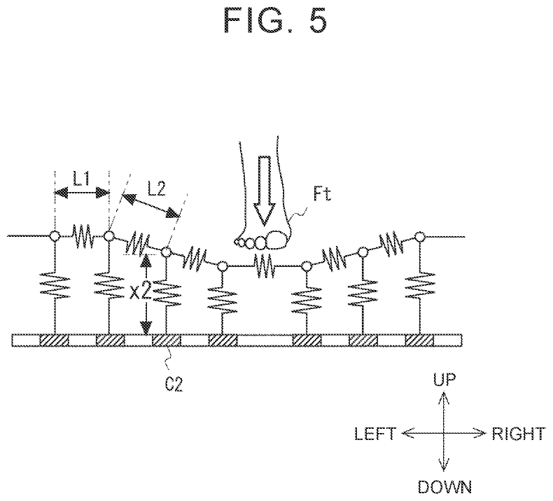

In FIG. 5, such a phenomenon is represented by a circuit. FIG. 5 is a front view of the region A for explaining the problem of the embodiment. When a load is applied from the sole Ft in the vertical direction and deformation due to the above-mentioned horizontal stress conversion occurs, the distance between adjacent measurement points changes. For example, a distance x2 between a cell C2 and the measurement point changes due to the extension of an edge L2 between the measurement points. When the distance x2 changes, an error occurs in the sensor output information and thus, the load distribution sensor 150 cannot acquire the correct load.

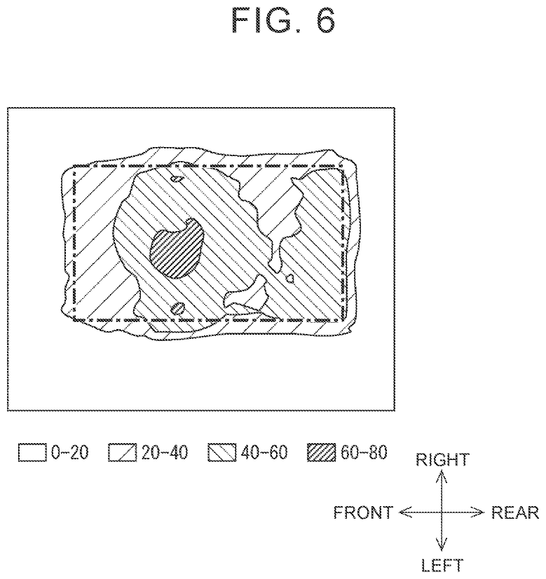

FIG. 6 is a diagram showing an example of the sensor output information. FIG. 6 visually shows the sensor output information of the load distribution sensor 150 when a weight of 90 kg is mounted on the belt 132 instead of the sole Ft as an example. The alternate long and short dash line in this FIG. 6 indicates the region (mounting region) on which the weight is actually placed. From FIG. 6, it can be seen that the load distribution sensor 150 detects the load in a range wider than the mounting region due to the deformation in the horizontal direction.

The present embodiment is for solving such a problem.

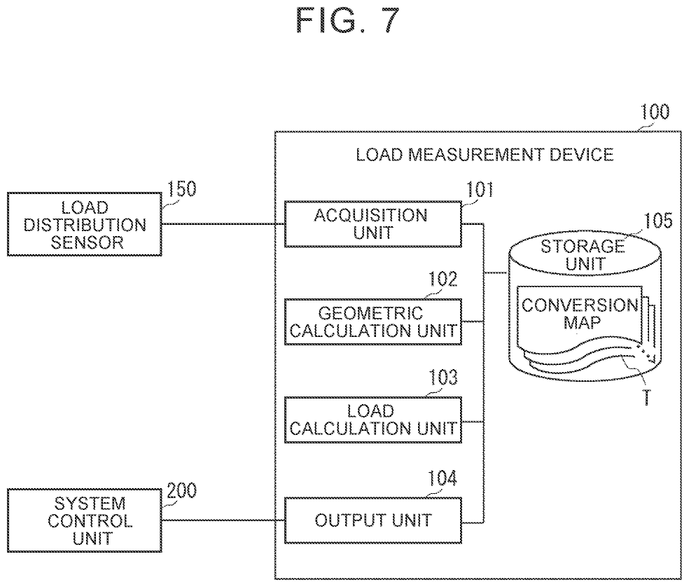

FIG. 7 is a block diagram showing a schematic configuration of the load measurement device 100 according to the first embodiment. The load measurement device 100 includes an acquisition unit 101, a geometric calculation unit 102, a load calculation unit 103, an output unit 104, and a storage unit 105. The components of the load measurement device 100 are connected to each other.

The acquisition unit 101 acquires the sensor output information output from the load distribution sensor. The sensor output information includes information on the output value of each of the cells having different positions from each other (that is, the voltage value corresponding to the pressure of each cell). The acquisition unit 101 may acquire the sensor output information from the load distribution sensor 150, or may acquire the sensor output information from another device (not shown) connected to the load distribution sensor 150. Then, the acquisition unit 101 supplies the sensor output information to the geometric calculation unit 102 and the load calculation unit 103.

The geometric calculation unit 102 specifies the load region SL based on the sensor output information, and calculates the geometric information of the specified load region SL. The geometric information includes at least the horizontal perimeter of the load region SL.

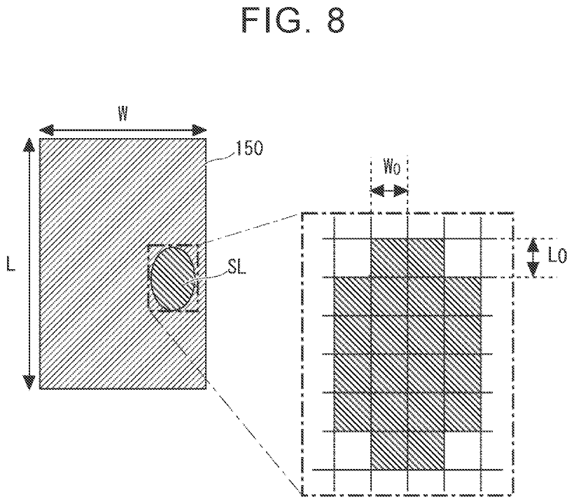

FIG. 8 is a diagram for specifically explaining the calculation process of the geometric information according to the first embodiment. First, from the sensor output information, the geometric calculation unit 102 specifies as the load region SL, a region in which the output value of the cell included in the load distribution sensor 150 is equal to or higher than a predetermined value. The load region SL is a set of cells having an output value equal to or higher than a predetermined value. As an example, one cell is a square having 1.3 cm of each side, that is, W₀=1.3 [cm] and L₀=1.3 [cm]. Thus, the geometric calculation unit 102 can calculate the perimeter of the load region SL by calculating the number of sides that are not adjacent to the other cells, among the four sides of the cell.

The geometric information may include the horizontal area of the load region SL in addition to the horizontal perimeter of the load region SL. In this case, the geometric calculation unit 102 can calculate the area by counting the number of cells having an output value equal to or higher than a predetermined value.

In this way, the geometric calculation unit 102 can easily calculate the geometric information of the load region SL based on geometric features such as the shape, number, area, and position information (particularly the positional relationship with the adjacent cells) of the cells in which the output value is equal to or higher than the predetermined value. The geometric calculation unit 102 supplies the calculated geometric information to the load calculation unit 103.

Returning to FIG. 7, the explanation will be continued. The load calculation unit 103 calculates the total load value based on the sensor output information and the geometric information. More specifically, the geometric calculation unit 102 selects a conversion map T based on the geometric information, and calculates the total load value based on the selected conversion map T and the sensor output information. As a result of diligent research, the inventors have found that the geometric information, especially the perimeter, correlates with the total load value. The load calculation unit 103 supplies the information on the total load value to the output unit 104.

The output unit 104 is connected to the system control unit 200. The output unit 104 outputs the total load value to the system control unit 200.

The storage unit 105 is a storage medium for storing information necessary for processing of the load measurement device 100 and the generated information. For example, the storage unit 105 stores the conversion map T associated with the geometric information. As an example, the storage unit 105 stores the conversion map T corresponding to the range of the perimeter.

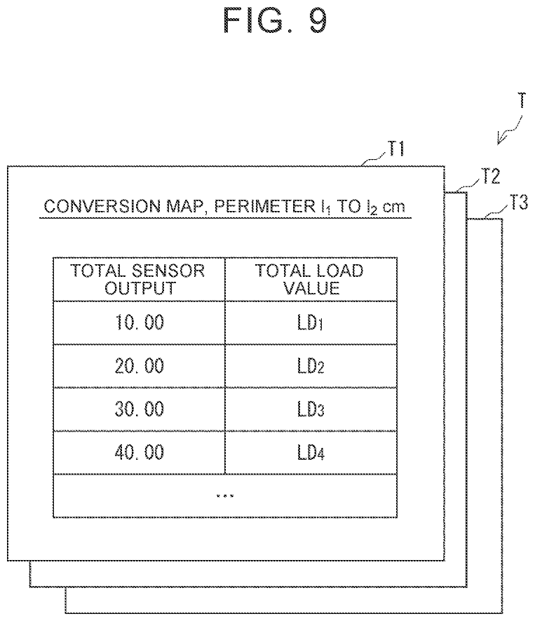

FIG. 9 is a diagram showing an example of the conversion map T according to the first embodiment. For example, a conversion map T1 is a conversion map that is selected by the load calculation unit 103 when the perimeter of the load region SL is between 1₁ and 1₂ (cm). As an example, the conversion map T1 is a map that associates the total sensor output with the total load value. The total sensor output may be the total of the output value of the cells included in the load distribution sensor 150. The output value of each cell is included in the sensor output information. That is, the conversion map T associates the geometric information with the total of the output value of the cell and the total load value. The load calculation unit 103 converts the sensor output information into the total load value by using the conversion map T1 that is selected based on the geometric information.

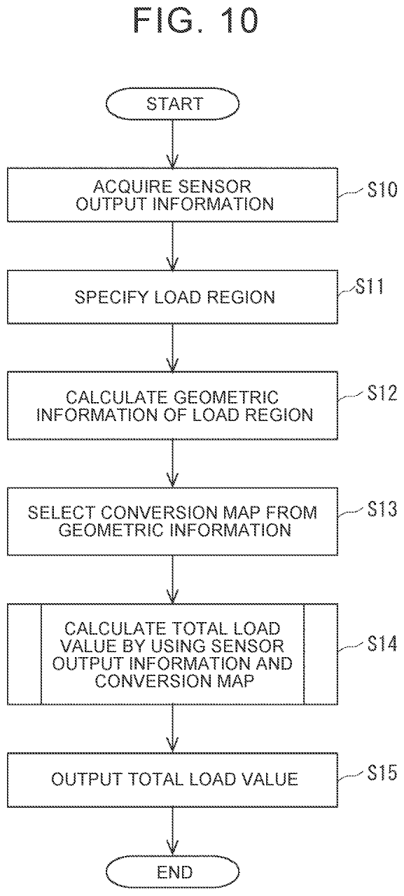

FIG. 10 is a flowchart showing a procedure of a load measurement method according to the first embodiment. First, the acquisition unit 101 acquires the sensor output information output by the load distribution sensor 150 (S10). Next, the geometric calculation unit 102 specifies the load region SL based on the output value of each cell included in the sensor output information (S11). Next, the geometric calculation unit 102 calculates the geometric information of the load region SL based on the geometric features of the cells included in the load region SL (S12). Next, the load calculation unit 103 selects the conversion map T corresponding to the geometric information, from the geometric information (S13). Then, the load calculation unit 103 calculates the total load value by using the sensor output information and the conversion map T (S14). For example, the load calculation unit 103 calculates the total of the output value of the cells included in the load region SL as the total sensor output, and specifies the total load value associated with the total sensor output in the selected conversion map T. As a result, the load calculation unit 103 can calculate the total load value. Next, the output unit 104 outputs the total load value to the system control unit 200 (S15). Then, for example, the system control unit 200 transmits a signal for controlling the extension of the walking assist device 120 to the auxiliary control unit 220 in response to the fact that the total load value becomes less than a predetermined threshold value.

As described above, according to the first embodiment, the load measurement device 100 can acquire the load value in the vertical direction in consideration of the load amount that has flowed in the horizontal direction due to the deformation in the horizontal direction at the time of load measurement. Therefore, the measurement accuracy of the load value is improved. As a result, the system control unit 200 can appropriately control the extension of the leg robot.

In the first embodiment, the total load value can be easily calculated while suppressing the calculation load, by converting the sensor output information into the total load value by using the conversion mapT. Such a method of calculating the total load value is effective in the walking training system 1 that requires real-time performance.

### Second Embodiment

Next, a second embodiment of the present disclosure will be described.

The load measurement device 100 according to the second embodiment basically has the same configuration and function as the load measurement device 100 according to the first embodiment. However, in the second embodiment, the load measurement device 100 is characterized in that the output value for each cell is converted into the pressure value for each cell by using the conversion map T, and the total load value is calculated based on the pressure value for each cell.

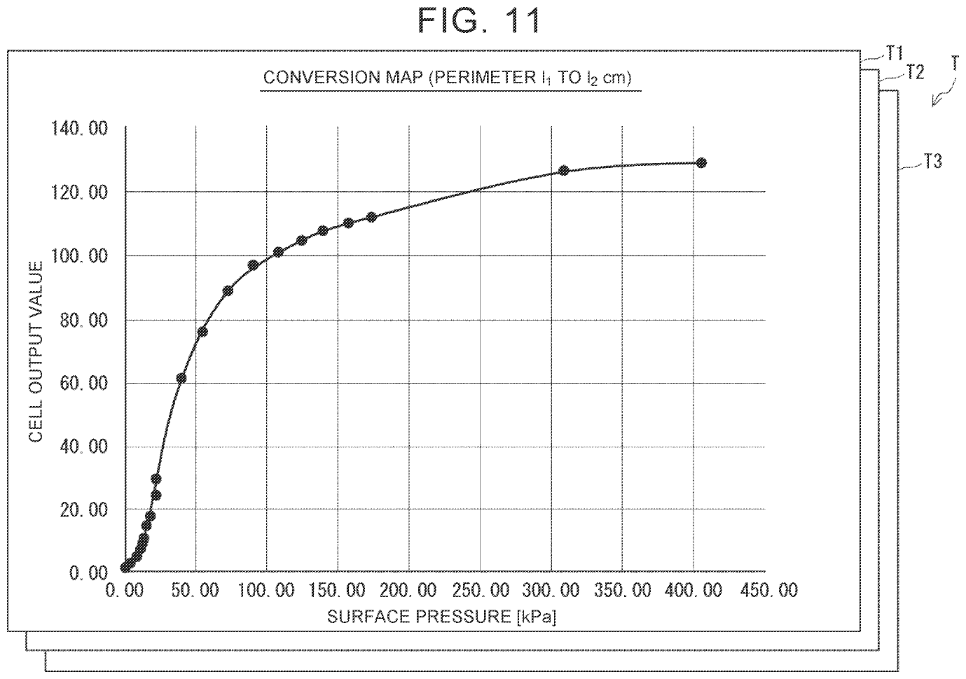

FIG. 11 is a diagram showing an example of the conversion map T according to the second embodiment. The conversion map T1 in FIG. 11 is a conversion map that is selected by the load calculation unit 103 when the perimeter of the load region SL is between 1₁ and 1₂ (cm). As an example, the conversion map T1 is a map that associates the output value of the cell with the surface pressure (pressure value). That is, the conversion map T associates the geometric information with the output value of the cell included in the sensor output information and with the pressure value. The load calculation unit 103 converts the output value for each cell included in the load distribution sensor 150 into the pressure value for each cell, by using the conversion map T1 that is selected based on the geometric information. Then, the load calculation unit 103 calculates the total load value based on the pressure value for each cell.

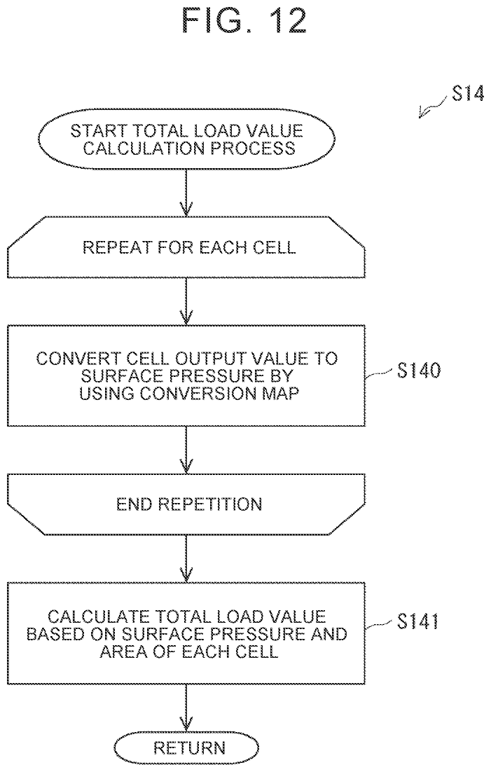

FIG. 12 is a flowchart showing the procedure of the total load value calculation process (that is, S14 in FIG. 10) in the load measurement method according to the second embodiment. First, the load calculation unit 103 repeats the process of converting the output value of the cell into the surface pressure for each cell included in the load region SL, by using the conversion map T that is selected based on the geometric information of the load region SL (S140). Then, the load calculation unit 103 calculates the total load value based on the surface pressure of each cell and the area per cell (S141). For example, the load calculation unit 103 may calculate the total load value by adding up the product of the surface pressure of each cell and the area per cell.

As described above, according to the second embodiment, the same effect as that of the first embodiment can be acquired. Further, according to the second embodiment, since the output value is converted into the pressure value one cell at a time, the load calculation accuracy is further improved.

### Third Embodiment

Next, a third embodiment of the present disclosure will be described.

The third embodiment is characterized in that a trained load estimation model is used instead of the conversion map T for calculating the total load value.

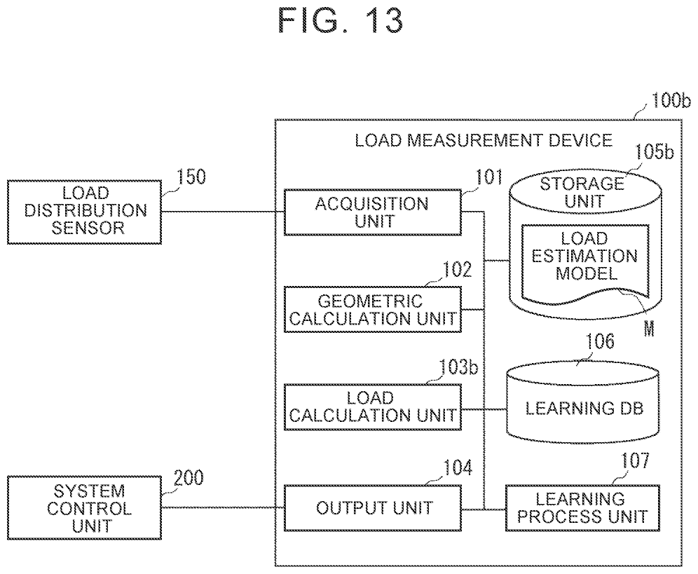

FIG. 13 is a block diagram showing a schematic configuration of a load measurement device 100b according to the third embodiment. The load measurement device 100b has basically the same configuration and function as the load measurement device 100. However, the load measurement device 100b is different from the load measurement device 100 in that the load measurement device 100b has a load calculation unit 103b, a storage unit 105b, a learning database (DB) 106, and a learning process unit 107 instead of the load calculation unit 103 and the storage unit 105.

The load calculation unit 103b calculates the total load value by using a trained load estimation model M. In the load estimation model M, the input is the sensor output value and the geometric information, and the output is the total load value. The sensor output value may be the cell output value in the load region SL or the sum of the cell output values (total sensor output), included in the sensor output information. For example, the load calculation unit 103b inputs the total sensor output and the load information of the load region SL into the load estimation model M, and acquires the total load value output from the load estimation model M. As a result, the load calculation unit 103b can easily estimate the total load value even for an unknown combination of the geometric information and the sensor output value.

The storage unit 105b stores the trained load estimation model M instead of the conversion map T.

The learning DB 106 is a database that stores teacher data including the sensor output value and the geometric information tagged with the total load value.

The learning process unit 107 optimizes the parameters of the load estimation model M by using the teacher data stored in the learning DB 106. As a result, the load estimation model M is learned.

The load estimation model M may be a model that outputs the total load value from a matrix diagram in which the output value of each cell is held as a pixel value. In this case, in the load estimation model M, the matrix diagram is an input and the total load value is the output. The load estimation model M may include, for example, a convolutional neural network (CNN). Then, the load calculation unit 103b may generate the matrix diagram based on the output value of each cell and the position information of each cell and input the matrix diagram to the load estimation model M, so as to acquire the total load value output from the load estimation model M. As a result, the load calculation unit 103b can easily and accurately estimate the total load value even if the geometric information is complicated. In this case, the geometric calculation unit 102 of the load measurement device 100b may be omitted.

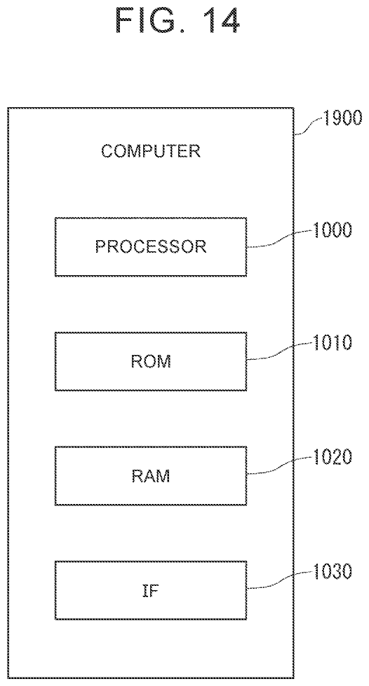

FIG. 14 is a schematic configuration diagram of a computer that is used as the load measurement devices 100, 100b and the system control unit 200 according to the first to third embodiments.

The computer 1900 includes a processor 1000, a read only memory (ROM) 1010, a random access memory (RAM) 1020, and an interface unit 1030 (IF; interface) as main hardware configurations. The processor 1000, the ROM 1010, the RAM 1020 and the interface unit 1030 are connected to each other via a data bus and the like.

The processor 1000 has a function as an arithmetic unit that performs a control process, an arithmetic process, and the like. The processor 1000 may be a central processing unit (CPU), a graphics processing unit (GPU), a field-programmable gate array (FPGA), a digital signal processor (DSP), an application specific integrated circuit (ASIC), or a combination thereof. The ROM 1010 has a function for storing a control program, an arithmetic program, and the like executed by the processor 1000. The RAM 1020 has a function for temporarily storing processing data and the like. The interface unit 1030 inputs and outputs signals to and from the outside by wire or wirelessly. The interface unit 1030 receives the operation of inputting data by the user and displays information to the user. For example, the interface unit 1030 communicates with the load distribution sensor 150, the input portion 142, and the system control unit 200.

In the above example, the program includes instructions (or software codes) for causing the computer to perform one or more of the functions described in the embodiments when loaded into the computer. The program may be stored on various non-transitory computer-readable media or tangible storage media as an example of the ROM 1010. Examples of the computer-readable media or tangible storage media include, but not limited to, a RAM, a ROM, a flash memory, a solid-stated rive (SSD) or other memory technologies, a compact disc (CD)-ROM, a digital versatile disc (DVD), a Blu-ray (registered trademark) disc, or other optical disc storages, a magnetic cassette, a magnetic tape, a magnetic disc storage, or other magnetic storage devices. The program may be transmitted on a transitory computer-readable medium or a communication medium. Examples of the transitory computer-readable medium or the communication medium include, but not limited to, electrical, optical, acoustic, or other forms of propagating signals.

In the above embodiments, the computer 1900 is composed of a computer system including a personal computer, a word processor, and the like. However, the present disclosure is not limited thereto, and the computer 1900 can be composed of a local area network (LAN) server, a host computer of computer (personal computer) communication, a computer system connected on the Internet, and the like. The computer 1900 can be composed of the network as a whole with the functions being distributed to various devices on the network. Thus, the components of the load measurement device 100 may be distributed in different devices.

The present disclosure is not limited to the above embodiments, and can be appropriately modified without departing from the scope thereof.

What is claimed is:

1. A load measurement system comprising:
an acquisition unit that acquires sensor output information of a load distribution sensor that detects a load distribution received from a sole of a subject;
a load calculation unit that calculates a total load value, based on the sensor output information and geometric information of a load region that is specified based on the sensor output information, the geometric information including at least a perimeter of the load region in a horizontal direction; and
an output unit that outputs the total load value.

2. The load measurement system according to claim 1, further comprising a geometric calculation unit that specifies a region in which an output value of a cell included in the load distribution sensor is equal to or higher than a predetermined value, and that calculates the geometric information of the load region.

3. The load measurement system according to claim 1, wherein the load calculation unit calculates the total load value by using a map that associates the geometric information with a total of an output value of a cell included in the load region and the total load value.

4. The load measurement system according to claim 1, wherein the load calculation unit uses a map that associates the geometric information with output values of cells included in the load region and pressure values to calculate a pressure value for each of the cells included in the load distribution sensor, and the load distribution sensor calculates the total load value based on the pressure value of each of the cells.

5. The load measurement system according to claim 1, wherein the load calculation unit calculates the total load value by using a trained load estimation model in which an input is an output value of a cell included in the load region, or a total of the output value of the cell, and in which an output is the total load value.

6. A walking training system comprising:
the load measurement system according to claim 1;
the load distribution sensor;
a moving body; and
a control device that controls extension of a leg robot attached to at least one leg of the subject, the subject walking on a walking surface provided on the moving body, based on the total load value.

7. A load measurement method comprising:
acquiring sensor output information of a load distribution sensor that detects a load distribution received from a sole of a subject;
calculating a total load value, based on the sensor output information and geometric information of a load region specified based on the sensor output information, the geometric information including at least a perimeter of the load region in a horizontal direction; and
outputting the total load value.

8. A program that causes a computer to execute:
an acquisition process of acquiring sensor output information of a load distribution sensor that detects a load distribution received from a sole of a subject;
a load calculation process of calculating a total load value, based on the sensor output information and geometric information of a load region specified based on the sensor output information, the geometric information including at least a perimeter of the load region in a horizontal direction; and
an output process of outputting the total load value.
